(19) United States
(12) Patent Application Publication
(10) Pub. No.: US 2009/0197855 A1
(43) Pub. Date: Aug. 6, 2009

(54) PHARMACEUTICAL COMPOSITIONS OF CEFDINIR

(76) Inventors: Makarand Krishnakumar Avachat, Maharashtra (IN); Sanjay Chhagan Wagh, Maharashtra (IN); Nital Arvind Jugade, Maharashtra (IN)

Correspondence Address: MERCHANT & GOULD PC, P.O. BOX 2903, MINNEAPOLIS, MN, 55402-0903, US

(21) Appl. No.: 12/299,280

(22) PCT Filed: Mar. 8, 2007
(86) PCT No.: PCT/IN07/00087

§ 371 Date: Mar. 25, 2009

(30) Foreign Application Priority Data
May 1, 2006 (IN) 388/KOL/2006

(51) Int. Cl. A61K 31/546; A61P 31/04

(52) U.S. Cl. 514/207

(57) ABSTRACT

A chewable tablet dosage form comprising Cefdinir or pharmaceutically acceptable salts thereof for the oral administration of Cefdinir in a manner that is more palatable and less objectionable to population group's especially young children and older patients. A method of preparing the chewable tablets comprising Cefdinir and method of treatment using the same.

### FIELD OF INVENTION

The present invention relates to pharmaceutical composition comprising Cefdinir or pharmaceutically acceptable salts thereof.

More specifically, the invention relates to a chewable tablet dosage form for the oral administration of Cefdinir in a manner that is more palatable and less objectionable to population group's especially young children and older patients.

Also disclosed are methods of preparing the chewable tablets comprising Cefdinir and method s of treatment using the same.

### BACKGROUND OF INVENTION

Cefdinir is a semi-synthetic cephalosporin antibiotic for oral administration. Chemically, Cefdinir is 7-[2-(2-aminothiazol-4-yl)-2-hydroxyiminoacetamido]-3-vinyl-3-cephem-4-carboxylic acid (syn isomer). This molecule was disclosed first time in U.S. Pat. No. 4,559,334.

U.S. Pat. No. 4,935,507 claims the novel crystalline Form (Crystal A) of Cefdinir and a process for the preparing the same.

It is a third generation cephalosporin antibiotic and has broader antibacterial spectrum over general gram positive and gram negative bacteria than other antibiotics for oral administration.

It has been reported that Cefdinir has an excellent antibacterial activity against Staphylococci and streptococci (J. Antibiotics, Vol. XLI, No 6,829, 1988 by Y. Inamoto, et al.

Cefdinir is active against a wide spectrum of bacteria, including Staphylococcus aureus, Streptopoccus pneumoniae, Streptococcus pyogenes, Hemophilus influenzae, Moraxella catarrhalis, E. coli, Klebsiella pneumoniae, and Proteus mirabilis.

Cefdinir is indicated for the treatment of Adults and Adolescents with Community acquired pneumonia, acute maxillary sinusitis, pharyngitis/tonsillitis and uncomplicated skin/skin structure infections. Also indicated for the treatment of pediatric patients with acute bacterial otitis media, pharyngitis/tonsillitis and uncomplicated skin/skin structure infections.

Cefdinir is currently available in number of different formulations, for instance as oral suspensions and tablets. Different formulations and different amounts of Cefdinir are provided for adults/adolescents and pediatric patients for example as capsule comprising 300 mg and as oral suspension comprising 125 mg/5 ml and 250 mg/5 ml.

Although suspensions are the common mode of administration of Cefdinir especially to the pediatric population, they suffer from other disadvantages such as limited shelf life and lack of accuracy of dose measurement. The bitter taste of many such medicaments is also a drawback. The bulky nature of the container often precludes ease of carriage and storage.

Thus a need exists for developing a formulation of Cefdinir, which does not suffer from the disadvantages of the suspension formulation as elaborated above.

Solid dosage forms that are swallowed such as tablets and capsules provide accurate dosage, avoid taste problems and are more amenable to being portable; but since they have to disintegrate in the gastrointestinal tract and the medicament has then to dissolve before it can be absorbed, absorption tends to be slower than from a suspension and may be less than complete leading to bio-inequivalence. Also, some patients have difficulty in swallowing tablets and capsules, and there is a practical limit to the size, and therefore the dose, that can be swallowed.

The problems encountered in taking such large dosage forms give an unnecessary sensation of oppression to patients on occasion of taking them. Improvements in their administration have thus been required.

This is particularly true for geriatric and pediatric patient populations.

Thus, the present invention attempts to provide a new dosage form with improved administrability comprising Cefdinir, which would have a bioavailability similar to that of a suspension comprising Cefdinir, but without the disadvantages of above available dosage forms.

### SUMMARY OF THE INVENTION

In its principle embodiment the present invention provides a chewable tablet composition of Cefdinir and method of producing the same.

### DETAILED DESCRIPTION OF THE INVENTION

In its principle embodiment the present invention provides a chewable tablet composition of Cefdinir or pharmaceutically acceptable salts thereof and methods of producing the same.

In another embodiment the present invention provides for a chewable tablet composition comprising greater than 5% by weight of Cefdinir.

In another embodiment the present invention provides for a chewable tablet composition comprising greater than 10% to 50% by weight of Cefdinir.

In another embodiment the present invention provides for chewable tablet composition of Cefdinir or pharmaceutically acceptable salts thereof having a disintegration time of not more than 2 minutes.

In another embodiment the present invention provides for a chewable tablet composition greater than 10% by weight Cefdinir;
a) a diluent;
b) a disintegrant; and
c) a lubricant.

The present invention also teaches a method of treating acute bacterial otitis media, pharyngitis and tonsillitis with a chewable tablet comprising the greater than 10% weight of Cefdinir.

In addition the present invention may optionally comprise further excipients known to those skilled in the art. These include diluents, binders, disintegrants, stabilizers, wetting agents, sweeteners, thickening agents, dispersing agents, flavoring agents, preservatives, coloring agents, lubricants and flow-aids and the like. One excipient can perform more than one function.

The chewable tablet compositions of the present invention preferably contain from 5 to 80%, more preferably from 10 to 60%, and most preferably from 15 to 40% by weight of the Cefdinir.

Diluents, which include but are not limited to sucrose, mannitol, xylitol, sugar potassium, aspartame, dextrose, fructose, saccharin, sodium saccharin, sorbitol and mixtures thereof can be used.

Diluent's of this invention can also serve other functions namely as a sweetener.

The diluents present are preferably in the range of 10 to 90% by weight of tablet. A preferred diluent of the present invention is mannitol.

Binders, which include, but are not limited to, alkylcelluloses such as methyl cellulose, hydroxyalkylcelluloses such as hydroxypropylcellulose, low substituted hydroxypropylcellulose and hydroxypropyl methylcellulose, sodium carboxymethylcellulose or mixtures thereof, pregelatinised maize starch or polyvinylpyrrolidone can be used.

The binders present are preferably in the range of 0.1 to 10% by weight of tablet. A preferred binder of the present invention is low substituted hydroxypropylcellulose.

Disintegrants, which include but are not limited to, crospovidone, sodium starch glycolate, starches such as maize starch and dried starch, croscarmellose sodium and cellulose products such as microcrystalline cellulose, microfine cellulose, low substituted hydroxypropylcellulose and the like, either used singly or in admixture can be used.

The disintegrants are preferably present in the range of 0.5 to 20% by weight of tablet. A preferred disintegrant of the present invention is crospovidone.

Stabilizers, which include but are not limited to, tribasic sodium phosphate, anhydrous sodium carbonate, glycine, citric acid and the like or mixtures thereof.

Wetting agents, which include, but are not limited to, surfactants, either singly or in admixture. Examples of surfactants include, but are not limited to, the polysorbates, sodium lauryl sulphate, poloxamers and the like.

The wetting agents are preferably present in the range of 0.01 to 5% by weight of tablet.

Sweeteners include, but are not limited to, natural sweeteners such as sugars e.g. fructose, glucose, sucrose, sugar alcohols such as mannitol, sorbitol or mixtures thereof and artificial sweeteners such as sodium saccharine, sodium cyclamate and aspartame. The sweetening agents are preferably present in the range of 10 to 90% by weight of tablet.

Dispersing agents include, but are not limited to, colloidal silicon dioxide and surfactants, wherein the surfactant is used alone or as an admixture with one or more surfactant. Combinations of colloidal silicon dioxide with one or more surfactants can also be used.

Flavoring agents, as used herein, refers to an agent or a mixture of agents that adds flavor to a mixture. Representative flavoring agents include, but are not limited to, art banana flavor, lemon mint, artificial strawberry flavor and artificial cream flavor. The flavoring agents present are preferably in the range of 0.5 to 5% by weight of tablet. A preferred flavoring agent of the present invention is lemon mint flavor.

Taste enhancing agents include, but are not limited to, sodium chloride, glycine, citric acid, tartaric acid and the like and mixtures thereof.

Preservatives include, but are not limited to, benzoic acid and sorbic acid and their salts, methyl paraben, butylparaben, propylparaben and the like.

Coloring agents include, but are not limited to, titanium dioxide pigments, lake colors and iron oxide pigments.

Lubricants include, but are not limited to, magnesium stearate, calcium stearate, zinc stearate, magnesium oxide, sodium stearyl fumarate, hydrogenated vegetable oil, sodium lauryl stearate, stearic acid, cornstarch, colloidal silicon dioxide, talc, and mixtures thereof.

A preferred lubricant of the present invention is magnesium stearate. Lubricants are present in from about 0.25% to about 6% by weight of tablet.

Tablets of the present invention may be prepared by conventional techniques for example wet granulation, compaction or direct compression.

The invention is illustrated with following examples.

### Example 1

| Sr. No | Name of Ingredient | mg/tab |
|---|---|---|
| 1. | Cefdinir | 100.00 |
| 2. | Mannitol DC | 230.50 |
| 3. | Magnesium stearate | 3.00 |
| 4. | L-HPC LH 11 | 11.00 |
| 5. | Crospovidone | 30.00 |
| | (Polyplasdone XL 10) | |
| 6. | Colloidal Silicon dioxide | 2.00 |
| | (Aerosil 200) | |
| 7. | Aspartame | 10.00 |
| 8. | Iron oxide yellow | 0.50 |
| 9. | Art Banana Flavor | 10.00 |
| 10. | Magnesium Stearate | 3.00 |
| | Total | 400.00 |

Procedure:
1. Sift Cefdinir and Mannitol DC through s.s.sieve and mix well.
2. Sift magnesium stearate through s.s.sieve and mix with step 1 blend.
3. Compact step 2 blend on roller compactor.
4. Sift and mill compacts to get granules.
5. Sift L-HPC LH 11, Polyplasdone XL 10, Aspartame, and Art Banana flavor through s.s.sieve. and mix well.
6. Sift color Iron oxide yellow through s.s.sieve.
7. Sift Magnesium stearate and Aerosol 200 through s.s.sieve.
8. Mix step 4, 5, 6 and 7 in octagonal blender.
9. Compress the blend of step 8 using Suitable round FFBE punch

### Example 2

| Sr. No | Name of Ingredient | mg/tab |
|---|---|---|
| 1. | Cefdinir | 150.00 |
| 2. | Mannitol DC | 345.75 |
| 3. | Magnesium stearate | 4.50 |
| 4. | L-HPC LH 11 | 16.50 |
| 5. | Crospovidone | 45.00 |
| | (Polyplasdone XL 10) | |
| 6. | Colloidal Silicon dioxide | 3.00 |
| | (Aerosil 200) | |
| 7. | Aspartame | 15.00 |
| 8. | Iron oxide yellow | 0.75 |
| 9. | Art Banana Flavor | 15.00 |
| 10. | Magnesium Stearate | 4.50 |
| | Total | 600.00 |

### Procedure:

Same as in Example 1.

### Example 3

| Sr. No | Name of Ingredient | mg/tab |
|---|---|---|
| 1. | Cefdinir | 200.00 |
| 2. | Mannitol DC | 461.00 |
| 3. | Magnesium stearate | 6.00 |
| 4. | L-HPC LH 11 | 22.00 |
| 5. | Crospovidone | 60.00 |
| | (Polyplasdone XL 10) | |
| 6. | Colloidal Silicon dioxide | 4.00 |
| | (Aerosil 200) | |
| 7. | Aspartame | 20.00 |
| 8. | Iron oxide yellow | 1.00 |
| 9. | Art Banana Flavor | 20.00 |
| 10. | Magnesium Stearate | 6.00 |
| | Total | 800.00 |

### Procedure:

Same as in Example 1.

### Example 4

| Sr. No | Name of Ingredient | mg/tab |
|---|---|---|
| 1. | Cefdinir | 300.00 |
| 2. | Mannitol DC | 691.50 |
| 3. | Magnesium stearate | 9.00 |
| 4. | L-HPC LH 11 | 33.00 |
| 5. | Crospovidone | 90.00 |
| | (Polyplasdone XL 10) | |
| 6. | Colloidal Silicon dioxide | 6.00 |
| | (Aerosil 200) | |
| 7. | Aspartame | 30.00 |
| 8. | Iron oxide yellow | 1.50 |
| 9. | Art Banana Flavor | 30.00 |
| 10. | Magnesium Stearate | 9.00 |
| | Total | 1200.00 |

### Procedure:

Same as in Example 1.

### Example 5

| Sr. No | Name of Ingredient | mg/tab |
|---|---|---|
| 1. | Cefdinir | 100.00 |
| 2. | Mannitol DC | 223.40 |
| 3. | Magnesium stearate | 3.00 |
| 4. | L-HPC LH 11 | 15.00 |
| 5. | Crospovidone | 30.00 |
| | (Polyplasdone XL 10) | |
| 6. | Colloidal Silicon dioxide | 3.00 |
| | (Aerosil 200) | |
| 7. | Aspartame | 10.00 |
| 8. | Iron oxide yellow | 0.60 |
| 9. | Flavor Peppermint | 3.32 |
| 10. | Flavour lemon | 8.68 |
| 11. | Magnesium Stearate | 3.00 |
| | Total | 400.00 |

### Procedure:

1. Sift Cefdinir and Mannitol DC through 30 mesh s.s.sieve and mix properly.
2. Sift magnesium stearate through 40 mesh s.s.sieve and mix with step 1 blend.
3. Compact step 2 blend on roller compactor.
4. Sift and mill compacts to get granules fraction 30—mesh s.s.sieve.60 mesh s.s.sieve. fraction about 75% of the blend taken for compaction.
5. Sift L-HPC LH 11, Polyplasdone XL 10, and Aspartame, through 40 mesh s.s.sieve. and mix properly.
6. Sift Iron oxide yellow, Flavour Mint, and Flavour Lemon, Crospovidone 100 mesh s.s.sieve.
7. Sift Magnesium Stearate and Colloidal Silicon Dioxide through 40 mesh s.s.sieve.
8. Mix step 4, 5, 6 and 7 in octagonal blender.
9. Compress the blend of step 8 using Suitable tooling.

| Sr. No | Name of Ingredient | mg/tab |
|---|---|---|
| 1. | Cefdinir | 150.00 |
| 2. | Mannitol DC | 335.10 |
| 3. | Magnesium stearate | 4.50 |
| 4. | L-HPC LH 11 | 22.50 |
| 5. | Crospovidone | 45.00 |
| | (Polyplasdone XL 10) | |
| 6. | Colloidal Silicon dioxide | 4.50 |
| | (Aerosil 200) | |
| 7. | Aspartame | 15.00 |
| 8. | Iron oxide yellow | 0.90 |
| 9. | Flavour Peppermint | 5.00 |
| 10. | Flavour lemon | 13.00 |
| 11. | Magnesium Stearate | 4.50 |
| | Total | 600.00 |

### Procedure:

Same as in Example 5.

### Example 7

| Sr. No | Name of Ingredient | mg/tab |
|---|---|---|
| 1. | Cefdinir | 200.00 |
| 2. | Mannitol DC | 446.80 |
| 3. | Magnesium stearate | 6.00 |
| 4. | L-HPC LH 11 | 30.00 |
| 5. | Crospovidone | 60.00 |
| | (Polyplasdone XL 10) | |
| 6. | Colloidal Silicon dioxide | 6.00 |
| | (Aerosil 200) | |
| 7. | Aspartame | 20.00 |
| 8. | Iron oxide yellow | 1.20 |
| 9. | Flavour Peppermint | 6.64 |
| 10. | Flavour lemon | 17.36 |
| 11. | Magnesium Stearate | 6.00 |
| | Total | 800.00 |

### Procedure:

Same as in Example 5.

### Example 8

| Sr. No | Name of Ingredient | mg/tab |
|---|---|---|
| 1. | Cefdinir | 300.00 |
| 2. | Mannitol DC | 670.25 |
| 3. | Magnesium stearate | 9.00 |
| 4. | L-HPC LH 11 | 45.00 |
| 5. | Crospovidone | 90.00 |
| | (Polyplasdone XL 10) | |
| 6. | Colloidal Silicon dioxide | 9.00 |
| | (Aerosil 200) | |
| 7. | Aspartame | 30.00 |
| 8. | Iron oxide yellow | 1.75 |
| 9. | Flavour Peppermint | 10.00 |
| 10. | Flavour lemon | 26.00 |
| 10. | Magnesium Stearate | 9.00 |
| | Total | 1200.00 |

### Procedure:

Same as in Example 5.

### Example 9

| Sr. No | Name of Ingredient | mg/tab |
|---|---|---|
| 1. | Cefdinir | 100.00 |
| 2. | Mannitol | 116.25 |
| | (Mannitol 60) | |
| 3. | Crospovidone | 10.00 |
| | (Polyplasdone XL 10) | |
| 4. | L-HPC LH 21 | 16.00 |
| 5. | Colloidal Silicon dioxide | 5.00 |
| | (Aerosil 200) | |
| 6. | Purified water | Q.S |
| 7. | Mannitol (Pearlitol | 116.25 |
| | SD200) | |
| 8. | Aspartame | 8.00 |
| 9. | Crospovidone | 10.00 |
| | (Polyplasdone XL 10) | |
| 10. | Iron oxide yellow | 0.50 |
| 11. | Flavour Peppermint | 3.32 |
| 10. | Flavour lemon | 8.68 |
| 10. | Magnesium Stearate | 6.00 |
| | Total | 400.00 |

### Procedure:

1. Sift Cefdinir, Mannitol 60, Crospovidone XL 10, Colloidal Silicon Dioxide through and mix well.
2. Dissolve L-HPC LH 21 in Water.
3. Granulate step 1 blend using step 2 binder
4. Dry Granules and sift.
5. Sift Mannitol SD 200 and Aspartame.
6. Sift Iron oxide yellow, Flavour Mint, Flavour Lemon, Crospovidone.
7. Mix step 4, 5 and 6 well in a blender.
8. Compress using suitable tooling.

### Example 10

| | | Strength |
|---|---|---|
| Sr. No | Name of Ingredient | 150 mg |
| 1. | Cefdinir | 150.00 |
| 2. | Mannitol (Mannitol 60) | 174.37 |
| 3. | Crospovidone | 15.00 |
| | (Polyplasdone XL 10) | |
| 4. | L-HPC LH 21 | 24.00 |
| 5. | Colloidal Silicon dioxide | 7.50 |
| | (Aerosil 200) | |
| 6. | Purified water | Q.S |
| 7. | Mannitol (Pearlitol | 174.37 |
| | SD200) | |
| 8. | Aspartame | 12.00 |
| 9. | Crospovidone | 15.00 |
| | (Polyplasdone XL 10) | |
| 10. | Iron oxide yellow | 0.75 |
| 11. | Flavour Peppermint | 5.00 |
| 10. | Flavour lemon | 13.00 |
| 10. | Magnesium Stearate | 9.00 |
| | Total | 600.00 |

### Procedure:

Same as in Example 9.

### Example 11

| Sr. No | Name of Ingredient | mg/tab |
|---|---|---|
| 1. | Cefdinir | 200.00 |
| 2. | Mannitol (Mannitol 60) | 232.50 |
| 3. | Crospovidone | 20.00 |
| | (Polyplasdone XL 10) | |
| 4. | L-HPC LH 21 | 32.00 |
| 5. | Colloidal Silicon dioxide | 10.00 |
| | (Aerosil 200) | |
| 6. | Purified water | Q.S |
| 7. | Mannitol (Pearlitol | 232.50 |
| | SD200) | |
| 8. | Aspartame | 16.00 |
| 9. | Crospovidone | 20.00 |
| | (Polyplasdone XL 10) | |
| 10. | Iron oxide yellow | 1.00 |
| 11. | Flavour Peppermint | 6.64 |
| 10. | Flavour lemon | 17.36 |
| 10. | Magnesium Stearate | 12.00 |
| | Total | 800.00 |

### Procedure:

Same as in Example 9.

### Example 12

| Sr. No | Name of Ingredient | mg/tab |
|---|---|---|
| 1. | Cefdinir | 300.00 |
| 2. | Mannitol (Mannitol 60) | 348.75 |
| 3. | Crospovidone | 30.00 |
| | (Polyplasdone XL 10) | |
| 4. | L-HPC LH 21 | 48.00 |
| 5. | Colloidal Silicon dioxide | 15.00 |
| | (Aerosil 200) | |
| 6. | Purified water | Q.S |
| 7. | Mannitol (Pearlitol | 348.75 |
| | SD200) | |
| 8. | Aspartame | 24.00 |
| 9. | Crospovidone | 30.00 |
| | (Polyplasdone XL 10) | |
| 10. | Iron oxide yellow | 1.50 |
| 11. | Flavour Peppermint | 10.00 |
| 10. | Flavour lemon | 26.00 |
| 10. | Magnesium Stearate | 18.00 |
| | Total | 1200.00 |

### Procedure:

Same as in Example 9.

### Example 13

| Sr. No | Name of Ingredient | mg/tab |
|---|---|---|
| 1. | Cefdinir | 100.00 |
| 2. | Mannitol | 112.20 |
| | (Mannitol 60) | |
| 3. | Crospovidone | 14.00 |
| | (Polyplasdone XL 10) | |
| 4. | L-HPC LH 21 | 16.00 |
| 5. | Colloidal Silicon dioxide | 5.00 |
| | (Aerosil 200) | |
| 6. | Purified water | Q.S |
| 7. | Mannitol (Pearlitol | 112.20 |
| | SD200) | |
| 8. | Aspartame | 8.00 |
| 9. | Crospovidone | 14.00 |
| | (Polyplasdone XL 10) | |
| 10. | Iron oxide yellow | 0.60 |
| 11. | Flavour Peppermint | 3.32 |
| 10. | Flavour lemon | 8.68 |
| 10. | Magnesium Stearate | 6.00 |
| | Total | 400.00 |

### Procedure:

1. Sift Cefdinir, Mannitol 60, Crospovidone XL 10, Colloidal Silicon Dioxide through 30 mesh s.s sieve and mix well.
2. Dissolve L-HPC LH 21 in Water.
3. Granulate step 1 blend using step 2 binder
4. Dry Granules and sift.
5. Sift Mannitol SD 200 and Aspartame.
6. Sift Iron oxide yellow, Flavour Mint, Flavour Lemon, Crospovidone through 100 mesh s.s sieve.
7. Mix step 4, 5 and 6 well in a blender.
8. Compress step 7 blend using suitable tooling.

### Example 14

| Sr. No | Name of Ingredient | mg/tab |
|---|---|---|
| 1. | Cefdinir | 150.00 |
| 2. | Mannitol | 168.30 |
| | (Mannitol 60) | |
| 3. | Crospovidone | 21.00 |
| | (Polyplasdone XL 10) | |
| 4. | L-HPC LH 21 | 24.00 |
| 5. | Colloidal Silicon dioxide | 7.50 |
| | (Aerosil 200) | |
| 6. | Purified water | Q.S |
| 7. | Mannitol (Pearlitol | 168.30 |
| | SD200) | |
| 8. | Aspartame | 12.00 |
| 9. | Crospovidone | 21.00 |
| | (Polyplasdone XL 10) | |
| 10. | Iron oxide yellow | 0.90 |
| 11. | Flavour Peppermint | 5.00 |
| 10. | Flavour lemon | 13.00 |
| 10. | Magnesium Stearate | 9.00 |
| | Total | 600.00 |

### Procedure:

Same as in Example 13.

### Example 15

| Sr. No | Name of Ingredient | mg/tab |
|---|---|---|
| 1. | Cefdinir | 200.00 |
| 2. | Mannitol | 224.40 |
| | (Mannitol 60) | |
| 3. | Crospovidone | 28.00 |
| | (Polyplasdone XL 10) | |
| 4. | L-HPC LH 21 | 32.00 |
| 5. | Colloidal Silicon dioxide | 10.00 |
| | (Aerosil 200) | |
| 6. | Purified water | Q.S |
| 7. | Mannitol (Pearlitol | 224.40 |
| | SD200) | |
| 8. | Aspartame | 16.00 |
| 9. | Crospovidone | 28.00 |
| | (Polyplasdone XL 10) | |
| 10. | Iron oxide yellow | 1.20 |
| 11. | Flavour Peppermint | 6.64 |
| 10. | Flavour lemon | 17.36 |
| 10. | Magnesium Stearate | 12.00 |
| | Total | 800.00 |

### Procedure:

Same as in Example 13.

### Example 16

| Sr. No | Name of Ingredient | mg/tab |
|---|---|---|
| 1. | Cefdinir | 300.00 |
| 2. | Mannitol (Mannitol 60) | 336.625 |
| 3. | Crospovidone | 42.00 |
| | (Polyplasdone XL 10) | |
| 4. | L-HPC LH 21 | 48.00 |
| 5. | Colloidal Silicon dioxide | 15.00 |
| | (Aerosil 200) | |
| 6. | Purified water | Q.S |
| 7. | Mannitol (Pearlitol SD200) | 336.625 |
| 8. | Aspartame | 24.00 |
| 9. | Crospovidone | 42.00 |
| | (Polyplasdone XL 10) | |
| 10. | Iron oxide yellow | 1.75 |
| 11. | Flavour Peppermint | 10.00 |
| 10. | Flavour lemon | 26.00 |
| 10. | Magnesium Stearate | 18.00 |
| | Total | 1200.00 |

### Procedure:

Same as in Example 13.

### Disintegration Time

The disintegration time evaluation was made in 1,000 ml of water (15°-25° C.) using a USP disintegration tester, but without using any disk, with 30 cycles per minute of basket ascending and descending.

Test Preparation A: Tablets produced in Example 1 to 4.
Test Preparation B: Tablets produced in Example 5 to 8.
Test Preparation C: Tablets produced in Example 9 to 12.
Test Preparation D: Tablets produced in Example 13 to 16.

### Test Results:

A: 1.06 minutes to 1.37 minutes
B: 1.13 minutes to 1.36 minutes
C: 1.03 minutes to 1.44 minutes
D: 1.08 minutes to 1.40 minutes

The disintegration test results indicate that the test preparations of Examples 1 to 16 of this invention each shows good disintegrability.

While the present invention has been particularly described, in conjunction with a specific preferred embodiment, it is evident that many alternatives, modifications and variations will be apparent to those skilled in the art in light of the foregoing description. It is therefore contemplated that the appended claims will embrace any alternatives, modifications and variations as falling within the true scope and spirit of the present invention.

1. A chewable tablet dosage form comprising Cefdinir or pharmaceutically acceptable salts thereof.

2. A chewable tablet dosage form comprising greater than 5% by weight of Cefdinir.

3. A chewable tablet dosage form comprising about 10% to 50% by weight of Cefdinir.

4. A chewable tablet dosage form of claim 1 having a disintegration time of not more than 2 minutes.

5. A chewable tablet dosage form comprising
a) greater than 10% by weight Cefdinir;
b) a diluent;
c) a disintegrant; and
d) a lubricant.

6. A chewable tablet composition of claim 5 wherein the diluent is selected from the group comprising of sucrose, mannitol, xylitol, sugar potassium, aspaitame, dextrose, fructose, saccharin, sorbitol, glucose, sodium saccharin and mixture thereof can be used.

7. A chewable tablet composition of claim 5 wherein the disintegrant is selected from the group comprising of crospovidone, Sodium starch glycolate, starches such as maize starch and dried starch, croscarmellose sodium and cellulose products such as microcrystalline cellulose, croscarmellose calcium, pregelatinized starch, microfine cellulose, low substituted hydroxypropylcellulose, either used singly or in admixture.

8. A chewable tablet composition of claim 5 further comprises a flavoring agent selected from the group consisting of lemon mint flavor, art banana flavor, artificial strawberry flavor and artificial cream flavor.

9. A chewable tablet composition of claim 5 wherein the lubricant is selected from the group comprising of magnesium stearate, calcium stearate, zinc stearate, stearic acid, magnesium oxide, sodium stearyl fumarate, hydrogenated vegetable oil, sodium lauryl stearate, cornstarch, colloidal silicon dioxide, talc, and mixtures thereof.

10. A method of treating acute bacterial otitis media, pharyngitis and tonsillitis with a chewable tablet comprising of Cefdinir or pharmaceutically acceptable salts thereof.
